(19) United States
(12) Patent Application Publication
(10) Pub. No.: US 2013/0267829 A1
(43) Pub. Date: Oct. 10, 2013

(54) APPARATUS FOR CT-MRI AND NUCLEAR HYBRID IMAGING, CROSS CALIBRATION, AND PERFORMANCE ASSESSMENT

(71) Applicants: Navdeep Ojha, Mayfield Village, OH (US); Michael Andrew Morich, Mentor, OH (US)

(72) Inventors: Navdeep Ojha, Mayfield Village, OH (US); Michael Andrew Morich, Mentor, OH (US)

(73) Assignee: KONINKLIJKE PHILIPS ELECTRONICS N.V., EINDHOVEN (NL)

(21) Appl. No.: 13/993,351

(22) PCT Filed: Dec. 15, 2011
(86) PCT No.: PCT/IB11/55693

§ 371 Date: Jun. 12, 2013

Related U.S. Application Data
(60) Provisional application No. 61/423,619, filed on Dec. 16, 2010.

(51) Int. Cl. A61B 5/00

(52) U.S. Cl. CPC A61B 5/0035
USPC 600/411; 250/336.1

(57) ABSTRACT

A multiple modality imaging system (10) includes a MR scanner (12) which defines an MR imaging region (18), a nuclear imaging scanner (26) which defines a nuclear imaging region (34), an CT scanner (36) which defines an CT imaging region (42). Each scanner (12, 26, 36) having a longitudinal axis along which a common patient support (46) moves linearly through the MR, nuclear, and CT imaging regions (18, 34, 42). A marker (130, 140, 150), for use with the system (10), includes a radio-isotope marker (132) which is imageable by the nuclear imaging scanner (26) and the CT scanner (36) surrounded by a flexible silicone MR marker (134) which is imageable by the MR scanner (12) and the CT scanner (36). A calibration phantom (162), for use with the image scanner (10), includes a plurality of the markers (130, 140, 150) supported by a common frame having a known and predictable geometry.

The present invention relates to the diagnostic imaging systems and methods. It finds particular application in conjunction cross-calibration, performance assessment, and image registration of multi-modality imaging systems combining MRI, CT, and one of PET or SPECT, but may find applicability in other diagnostic or treatment systems.

In multi-modality imaging systems, two different sensing modalities, such as nuclear imaging scanners like PET or SPECT coupled with an anatomical scanner such as CT, XCT, MRI, and the like are used to locate or measure different constituents in the object space. For example, the PET and SPECT scanners create functional images indicative of metabolic activity in the body, rather than creating images of surrounding anatomy. CT scans allow doctors to see hard tissue internal structures such as bones within the human body; while MRI scans visualize soft tissue structures like the brain, spine, vasculature, joints, and the like. In MR scans, the nuclear proton spins of the body tissue, or other MR nuclei of interest, to be examined are aligned by a static main magnetic field B₀ and are excited by transverse magnetic fields B₁ oscillating in the radiofrequency (RF) band. The resulting relaxation signals are exposed to gradient magnetic fields to localize the resultant resonance. The relaxation signals are received by an RF coil and the data is reconstructed into a single or multiple dimension image. Software fusion of the anatomical data from either the MR or CT scan with the metabolic data from the PET/SPECT scan in a composite image gives physicians visual information to determine if disease is present, the location and extent of disease, and to track how rapidly it is spreading.

In PET scans, a patient is administered a radiopharmaceutical, in which the radioactive decay events of the radiopharmaceutical produce positrons. Each positron interacts with an electron to produce a positron-electron annihilation event that emits two oppositely directed gamma rays. Using coincidence detection circuitry, a ring array of radiation detectors surrounding the patient detects the coincident oppositely directed gamma ray events which correspond to the annihilation event. A line of response (LOR) connecting the two coincident detections contains the position of the annihilation event. The lines of response are analogous to projection data and are reconstructed to produce a two- or three-dimensional image.

A CT scan can also be used for attenuation correction further enhancing PET/SPECT images rather than just providing anatomical information. Attenuation correction in traditional nuclear scanners involves a transmission scan in which an external radioactive source rotates around the FOV of the patient and measures the attenuation through the examination region when the patient is absent and when the patient is present. The ratio of the two values is used to correct for non-uniform densities which can cause image artifacts and can mask vital features.

Hybrid PET/MR and SPECT/MR imaging systems offer simultaneous or consecutive acquisition during a single imaging session and promise to bridge the gap between anatomical imaging and biochemical or metabolic imaging. Integration of the anatomical data from either the MR or CT scan with the metabolic data from the PET/SPECT scan in a composite image gives physicians visual information to determine if disease is present, the location and extent of disease, and to track how rapidly it is spreading. However, there exists a need for a multiple modality imaging system which includes an MR, nuclear, and CT scanner which can provide composite images of hard tissue, soft tissue, and metabolic activity in a single imaging session.

A problem with multiple modality imaging systems is image registration between the modalities and RF or magnetic interference between scanners. Although positioning the patient in the same position for more than one exam by moving the patient a known longitudinal distance reduces the possibility of misregistration of images stemming from patient movement, there remains the possibility of misregistration due to mechanical misalignments between the imaging regions, and the like.

The present application provides a new and improved apparatus and method which overcomes the above-referenced problems and others.

In accordance with one aspect, a multiple modality imaging system is presented. The imaging system includes an MR scanner which defines an MR imaging region which receives a subject along an MR longitudinal axis, a nuclear imaging scanner which defines a nuclear imaging region which receives the subject along a nuclear longitudinal axis, and an x-ray computed tomography (XCT) scanner which defines an XCT imaging region which receives the subject along an XCT longitudinal axis. The MR, nuclear, and XCT longitudinal axes are aligned with one another. A common patient support moves linearly through the MR, nuclear, and XCT imaging regions.

In accordance with another aspect, a method of using multiple modality imaging system is presented. The scanner comprises an MR scanner which defines an MR imaging region, a nuclear imaging scanner which defines a nuclear imaging region, and an x-ray computed tomography (XCT) scanner which defines an XCT imaging region. The method includes positioning a subject on a common patient support which moves linearly through the MR, nuclear, and XCT imaging regions. The subject is moved linearly into the MR imaging region and MR image data is acquired. The subject is moved linearly into the nuclear imaging region and nuclear image data is acquired. The subject is moved linearly into the XCT imaging region and XCT image data is acquired.

In accordance of another aspect, an imaging system is presented. The imaging system includes a MR scanner which defines an MR imaging region, a nuclear imaging scanner which defines a nuclear imaging region, and a flat panel CT scanner which defines a CT imaging region. The MR, nuclear, and CT imaging regions share a common longitudinal axis along which a common patient support moves linearly between the three imaging regions. The system includes a gantry track along which the nuclear image scanner and the CT scanner linearly translate to form a closed arrangement between the MR scanner, nuclear scanner, and flat panel CT scanner to reduce a transit time and transit distance of the common patient support between the MR, nuclear, and CT imaging regions.

One advantage resides in that image registration errors are reduced.

Another advantage resides in that workflow is improved.

Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

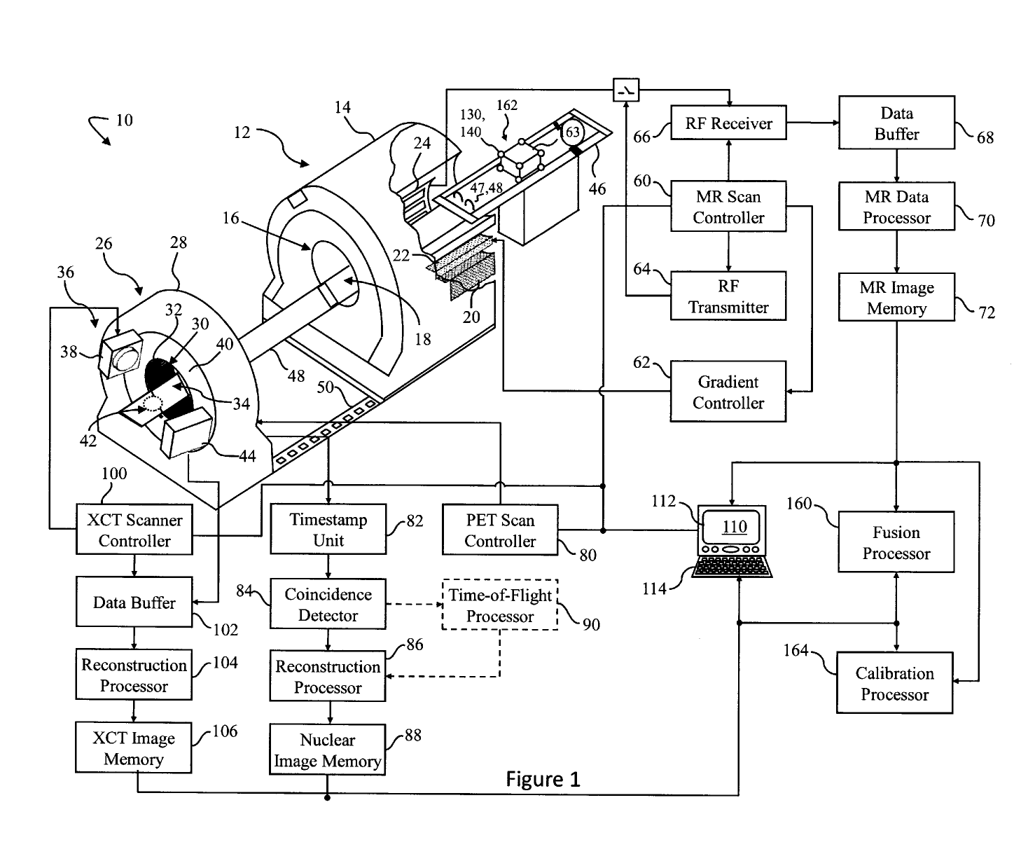

FIG. 1 is a diagrammatic illustration of a multiple modality imaging system and calibration processor;

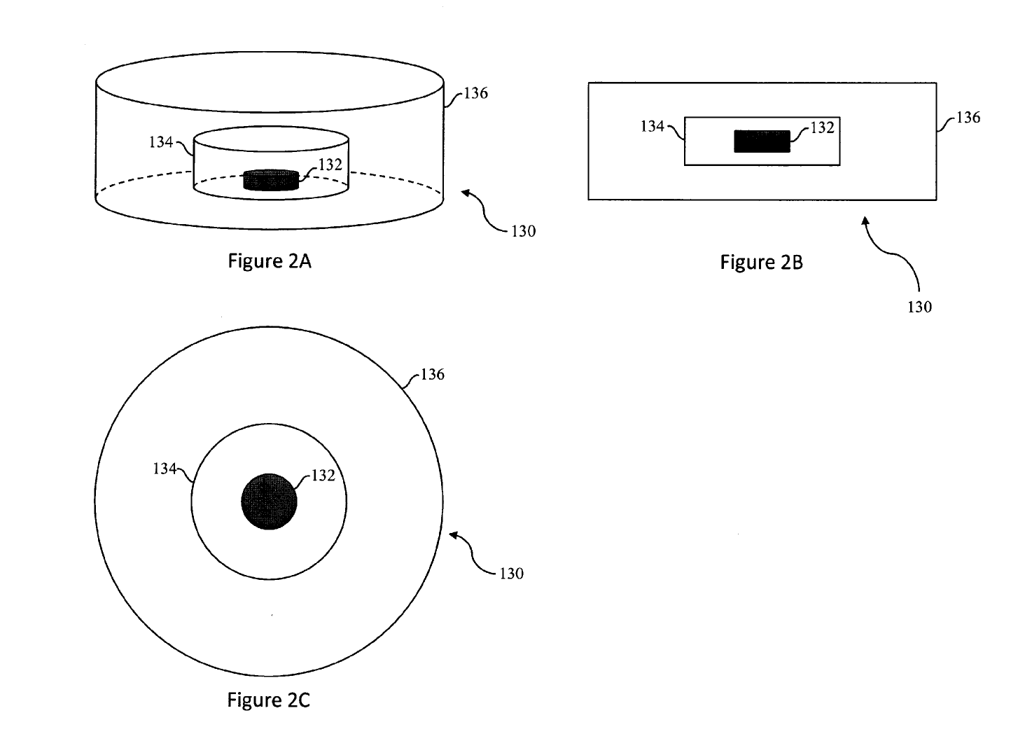

FIG. 2A is an isometric view of one embodiment of a multiple modality fiducial marker and FIGS. 2B and 2B are a side view and a top view, respectively;

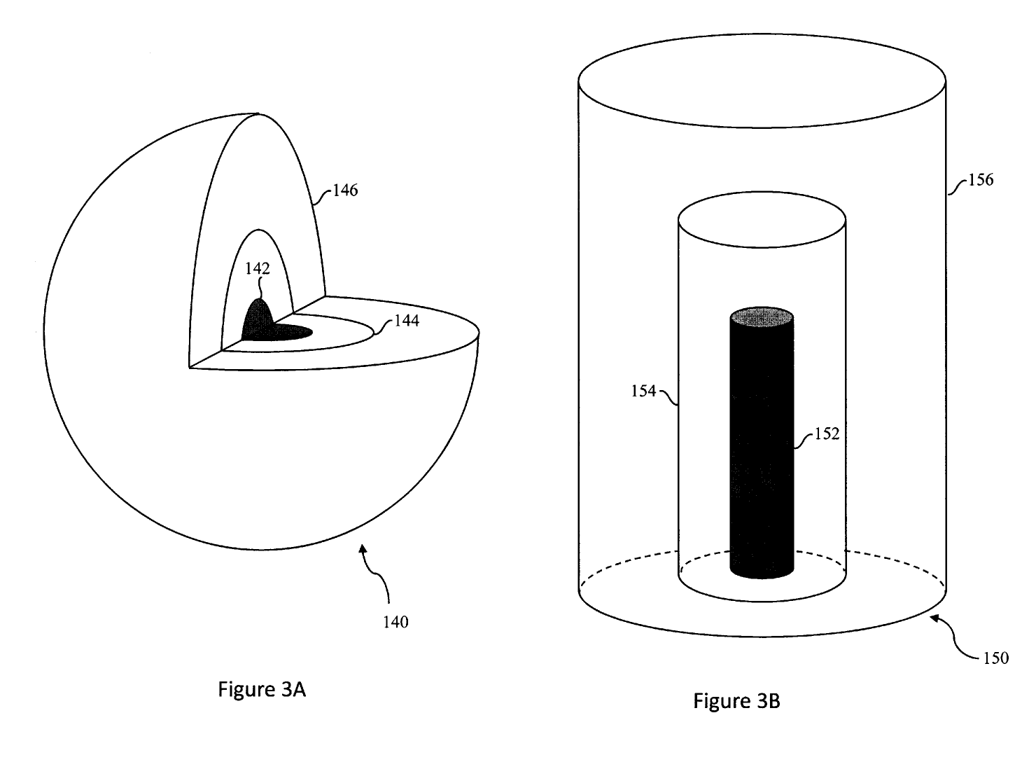

FIG. 3A is an isometric view in partial section of another embodiment of the multiple modality fiducial marker;

FIG. 3B is a diagrammatic illustration of another embodiment of the multiple modality fiducial marker;

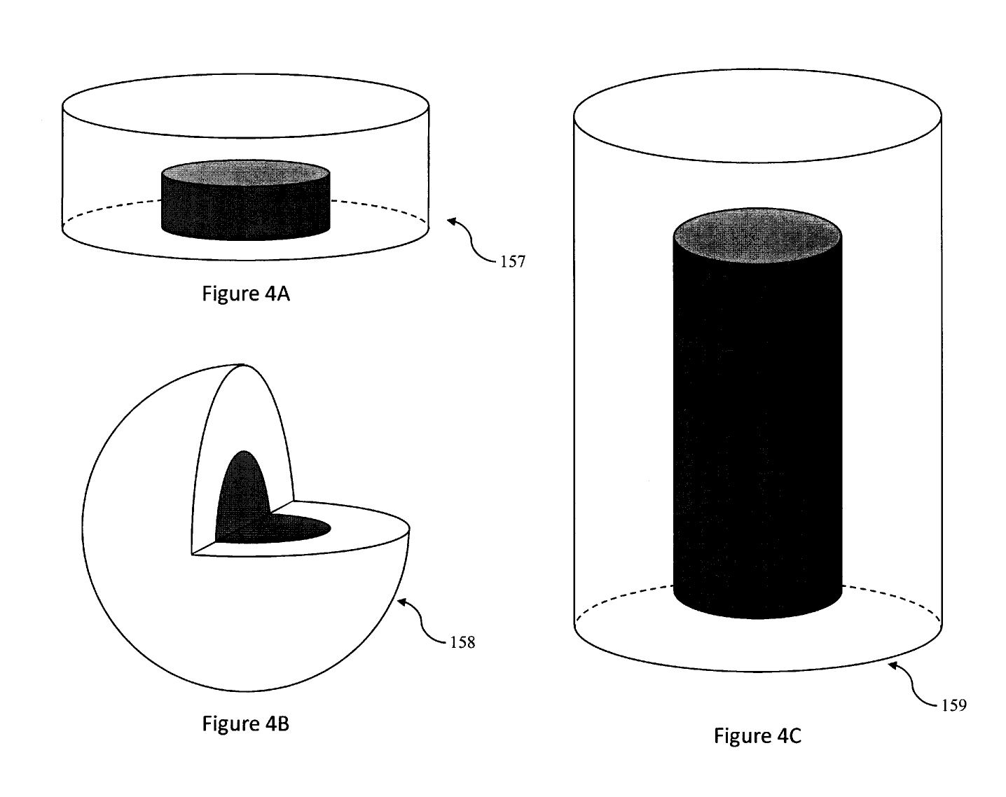

FIGS. 4A-4C are views of further embodiments of the multiple modality fiducial markers of FIGS. 2A-2C and FIGS. 3A-3B;

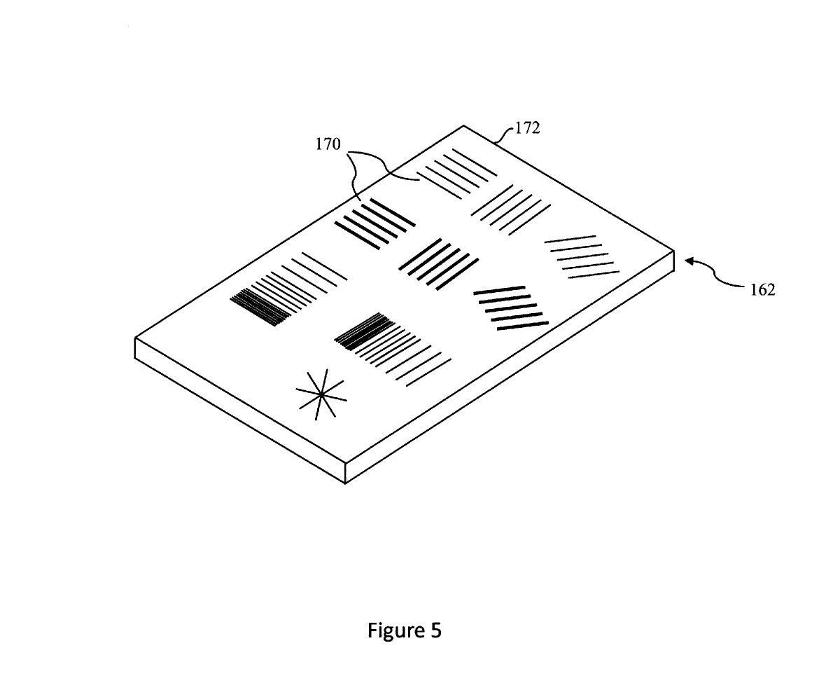

FIG. 5 is a diagrammatic illustration of an embodiment of a calibration phantom which includes one or more embodiments of the multiple modality marker;

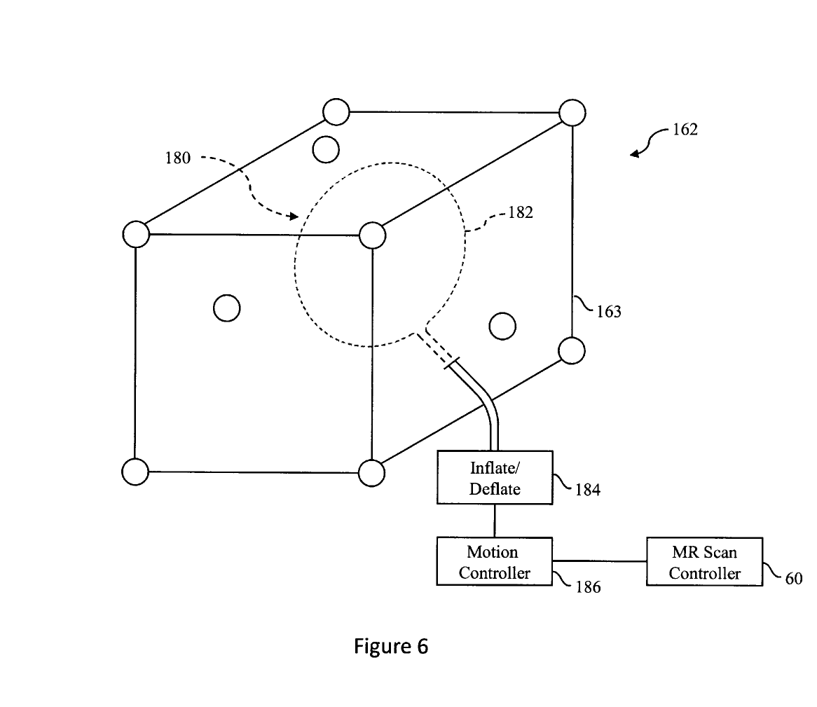

FIG. 6 illustrates a calibration phantom which simulates physiological motion; and

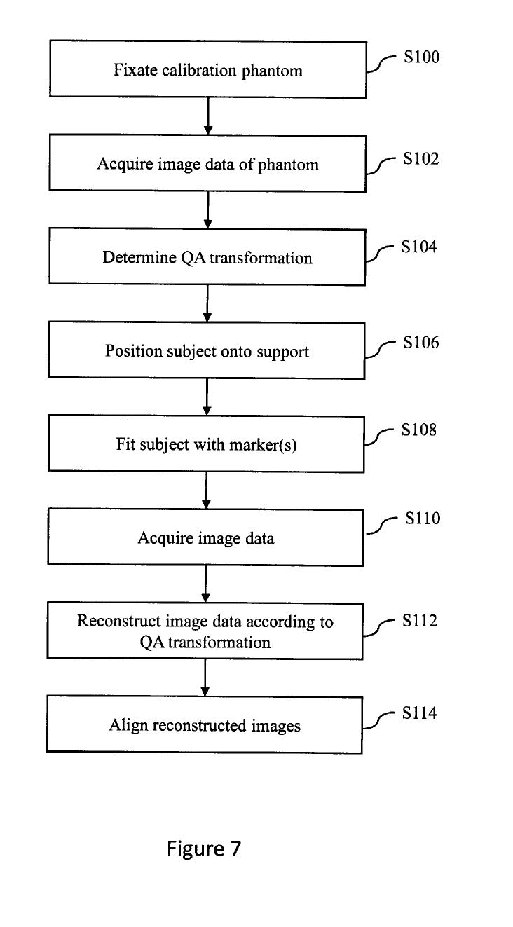

FIG. 7 is a flow chart of a method of calibrating the diagnostic imaging system of FIG. 1.

With reference to FIG. 1, a diagnostic imaging system 10 performs x-ray computer tomography (CT) and nuclear imaging, such as PET or SPECT, and magnetic resonance imaging and/or spectroscopy. The diagnostic imaging system 10 includes a first imaging system, in the illustrated embodiment a magnetic resonance scanner 12, housed within a first gantry 14. A first patient receiving bore 16 defines a first or MR examination region 18 of the MR scanner 12. The MR scanner includes a main magnet 20 which generates a temporally uniform B₀ field through the first examination region 18. Gradient magnetic field coils 22 disposed adjacent the main magnet serve to generate magnetic field gradients along selected axes relative to the B₀ magnetic field for spatially encoding magnetic resonance signals, for producing magnetization-spoiling field gradients, or the like. The magnetic field gradient coil 22 may include coil segments configured to produce magnetic field gradients in three orthogonal directions, typically longitudinal or z, transverse or x, and vertical or y-directions. A radio-frequency (RF) coil assembly 24, such as a whole-body radio frequency coil, is disposed adjacent the examination region. The RF coil assembly generates radio frequency B₁ pulses for exciting magnetic resonance in the aligned dipoles of the subject. The radio frequency coil assembly 24, or separate local receive-only RF coil (not shown) in addition to RF coil assembly 24, also serves to detect magnetic resonance signals emanating from the imaging region.

A second imaging system, in the illustrated embodiment a PET scanner 26, is housed within a second gantry 28 which defines a second patient receiving bore 30. It should be appreciated that a SPECT scanner is also contemplated. A stationary ring of radiation detectors 32 are arranged around the bore 30 to define a second or nuclear, particularly PET, examination region 34. In a SPECT scanner, the detectors 32 are incorporated into individual heads, which are mounted for rotation and radial movement relative to the subject.

A third imaging system, in the illustrated embodiment a CT scanner 36, such as a flat panel XCT scanner as illustrated and a conventional bore type scanners, includes an x-ray source 38 mounted on a rotating gantry 40 which rotates about the longitudinal axis of the bore 30 of the second gantry 28. The x-ray source 38 produces x-rays, e.g. a cone beam, passing through a third or CT examination region 42, where they interact with a target area of a subject (not shown) within the CT examination region 42. An x-ray detector array 44, such as a flat panel detector, is arranged opposite the examination region 42 to receive the x-ray beams after they pass through the examination region 42 where they interact with and are partially absorbed by the subject and a common patient support 46 and corresponding mechanical structures. The detected x-rays therefore include absorption information relating to the subject and the subject support mechanical structures. Where accessories 47, such as MR imaging accessories like local RF coils, RTP accessories like as fixation devices, or interventional devices, are also attached to the subject, the CT examination likewise provides attenuation information for the accessories.

The two gantries 14, 28 are adjacent to one another in a linear arrangement and in close proximity to one another. The gantries 14, 28 share a common patient support 46 that translates along a longitudinal axis between the three examination regions 18, 34, 42 along a patient support track or path 49. A motor or other drive mechanism (not shown) provides the longitudinal movement and vertical adjustments of the support in the examination regions 18, 34, 42. In the illustrated embodiment, the PET gantry 28 translates along a gantry track 50 to reduce the transit time between the three imaging systems 12, 26, 36. A close arrangement between gantries reduces the likelihood of patient movement and misregistration errors stemming from longer transit between the imaging systems 12, 26, 36. The gantries can be separated and related electronic systems can be selectively powered down to reduce interference between the imaging modalities. For example, the radiation detectors 32 and corresponding detection circuitry of the PET scanner 26 emit RF signals which may interfere with resonance detection of the MR scanner 12. RF shielding and filtering, selective electronics shut down, and temporarily increased distance between scanners are mitigation measures. Once an MR imaging procedure has concluded, the gantries can be arranged closer for patient relocation to the PET examination region 34 or the CT examination region 42 so as to reduce positioning errors. It is to be appreciated that the scanners may be in a nominally fixed relationship and/or utilize a patient support that is rotatable in the space between scanners. Also, the magnetic field sensitive portions of PET, SPECT and/or XCT/CT systems may be magnetically shielded to mitigate effects from the MR fringe magnetic field.

To acquire magnetic resonance data of a subject, the subject is positioned inside the MR examination region 18, preferably at or near an isocenter of the main magnetic field. A scan controller 60 controls a gradient controller 62 which causes the gradient coils 22 to apply the selected magnetic field gradient pulses across the imaging region, as may be appropriate to a selected magnetic resonance imaging or spectroscopy sequence. The scan controller 20 controls an RF transmitter 64 which causes the RF coil assembly to generate magnetic resonance excitation and manipulation B₁ pulses. The scan controller also controls an RF receiver 66 which is connected to the RF coil assembly 24 to receive the generated magnetic resonance signals therefrom. The received data from the receivers 68 is temporarily stored in a data buffer 68 and processed by a MR data processor 70. The MR data processor 70 can perform various functions as are known in the art, including image reconstruction (MRI), magnetic resonance spectroscopy (MRS), and the like. Reconstructed magnetic resonance images, spectroscopy readouts, and other processed MR data are stored in an MR image memory 72.

To acquire nuclear imaging data, the patient is re-positioned, particularly linearly translated, from the MR examination region 18 to the PET examination region 34 along the patient support track 49. The PET scanner 26 is operated by a PET scan controller 80 to perform selected imaging sequences of the selected target area. Typically, an object or patient to be imaged is injected with one or more radiopharmaceutical or radioisotope tracers then placed in the PET or SPECT examination region 34. Examples of such tracers for PET are 18F FDG, C-11, and for SPECT are Tc-99m, Ga67, and In-111. For SPECT tracers, gamma radiation is produced directly by the tracer. For PET, the presence of the tracer within the object produces emission radiation, particularly positron annihilation events which each produce a pair of γ rays travelling in opposite directions, from the object. Radiation events are detected by the radiation detectors 32 around the examination region 34. A time stamp is associated with each detected radiation event by a time stamp unit 82. A coincidence detector 84 determines coincident pairs of the γ rays and the line of responses (LOR) defined by each coincident pair of γ rays based on differences in detection time of the coincidence pairs and the known diameter of the field of view. A reconstruction processor 86 reconstructs the LORs into an image representation which is stored in a functional image memory 88. Optionally, a time-of-flight processor 90 localizes each radiation event along each LOR by deriving time-of-flight information from the timestamps.

To acquire CT data, the patient is re-positioned, e.g. linearly translated, from the PET examination region 34 to the CT examination region 42 along the patient support path 48. The CT scanner 36 is operated by a CT scan controller 100 to perform selected imaging sequences of a selected target area. The CT scan controller 100 controls the radiation source 38 and the rotating gantry 40 to traverse the CT examination region 42. The radiation detector 44 receives the x-ray data after passing through the subject which is then stored in a data buffer 102. A reconstruction processor 104 reconstructs an image representation from the acquired x-ray data, and the reconstructed image representations are stored in an CT image memory 106. In another embodiment, prior to acquiring the nuclear imaging data, the patient is positioned in the CT scanner 36 to acquire transmission data to generate an attenuation map. After the x-ray data in received, the CT reconstruction processor 104 generates an attenuation map which is then used by the PET reconstruction processor 86 to generate attenuation corrected image representations.

The diagnostic imaging system 10 includes a workstation or graphic user interface 110 which includes a display device 112 and a user input device 114 which a clinician can use to select scanning sequences and protocols, display image data, and the like.

With reference to FIGS. 2A-2C, in one embodiment, the patient, the patient support 46, or another article associated with the patient is outfitted with one or more of fiducial markers 130 which are imageable in all three imaging modalities, i.e. each are detectable by the MR scanner 12, the nuclear imaging scanner 26, and the CT scanner 36. Each fiducial marker 130 includes a radio-isotope marker 132 which is imageable by both the nuclear imaging scanner 26 and the CT scanner 36. The radio-isotope marker 132 can be a solid or an encapsulated liquid. Compatible PET imageable radio-isotopes include Na-22 and Ge-68. Compatible SPECT imageable radio-isotopes includes Co-57, Gd-153, Ce-139, Cd-109, Am-241, Cs-137, and Ba-133.

The radio-isotope marker 132 is surrounded by a MR marker 134 which is imageable by both the MR scanner 12 and the CT scanner 36. The MR marker 134 is a silicone rubber disk when cured is somewhat flexible so a rigid housing 136, such as acrylic, is placed around the radio-isotope marker 132 and MR marker 134 assembly. Both the radio-isotope marker 132 and MR marker 134 share a common center of mass or centroid in the respective image representation. Alternatively, the radio-isotope marker 132 and MR marker 134 have a fixed geometric relationship between their respective centroids. With reference to FIG. 3A, in another embodiment the fiducial markers 140 are shaped as spheres with a spherical radio-isotope marker 142, as a solid or liquid filled capsule, surround by a MR marker sphere 144, as silicone rubber sphere, and encased in a rigid housing 146. With reference to FIG. 3B, the fiducial markers 150 are shaped as cylinders with a cylindrical radio-isotope marker 152, as a solid or liquid filled capsule, surround by a MR marker cylinder 154, as silicone rubber cylinder, and encased in a rigid housing 156. Similarly, the radio-isotope marker 142, 152 and MR marker 144, 154 share a common center of mass or centroid or a fixed geometric relationship between their respective centroids.

With reference to FIGS. 4A-4C, in another embodiment, the radio-isotope is mixed with the silicone rubber to form a composite fiducial marker which is imageable by the MR, nuclear, and CT scanners 12, 26, 36. The radio-isotope, as a liquid or a powdered solid, is substantially uniformly dispersed throughout the silicone rubber while it is still in a liquid form prior to curing. In this arrangement, the composite fiducial markers 157, 158, 159 can take various shapes and geometries, such as a sphere, disk, cylinder or the like.

With reference to FIG. 1, the diagnostic imaging system 10 includes a fusion processor 160 which combines images from the MR scanner 12, the nuclear imaging scanner 26, and the CT scanner 36 to form a composite image representation of the subject. The fusion processor 160 receives the image representations from the respective image memories 72, 88, 106 and determines coordinates for the three-dimensional centroid of each fiducial marker 130, 140 positioned on the patient, near the patient, and/or on the patient support 46 in each image representation. The fiducials can be positioned on the table before patient imaging starts to align the table to each imaging system. The fusion processor 160 generates a fusion transformation which registers the three image representations into alignment based on the centroid coordinates. The fusion transformation includes translating, scaling, rotating, and the like such that the MR image representation, nuclear image representation, and the CT image representation are accurately registered to one another. In this arrangement, image representations acquired in the same imaging session, i.e. the subject remaining on the patient support during MR, nuclear, and CT acquisition, can be merged and co-registered with minimal patient movement and misregistration errors. The result is a composite image which visualizes soft tissue structures, metabolic activity, and hard tissue structures.

In one embodiment, the diagnostic imaging system 10 includes a calibration phantom 162 for calibration of the three image scanners, the MR scanner 12, the nuclear scanner 26, and the CT scanner 36, to verify resolution, distortions, uniformity, contrast to noise ratio, contrast recovery, background noise, and the like. The calibration phantom 162 includes at least one fiducial marker 130, 140 arranged in and supported by a common imaging frame 163 which has a known and predictable shape, geometry, or structure. The number of fiducial markers 130, 140 arranged in the frame is dependent on the application. In the illustrated embodiment, the imaging frame 163 is a cube with the fiducial markers 130, 140 positioned at each of the eight corners. Various shapes, geometries with varying spacings, and complex structures are also contemplated.

In another embodiment shown in FIG. 5, the calibration phantom 162 has at least one pattern 170 with a plurality of lines of the silicone rubber mixed or embedded with the radio-isotope, as described with reference to FIGS. 4A-4C, supported by a flat, rigid housing or sheet 172, particularly of acrylic. Each pattern 170 includes an array or sets of lines having varying widths, spacings, and orientations to test for and quantify resolution characteristics in different directions of each of the image scanners 12, 26, 36.

After the phantom 162 is rigidly mounted or affixed to the patient support 46, the user selects a calibration sequence via the user interface 110 and the diagnostic imaging system 10 positions the phantom 162 in the respective examination regions 18, 34, 42 for data acquisition. The corresponding scanner controllers 60, 80, 100 control the respective scanners 12, 26, 36 to acquire 3D imaging data of the phantom 162. The imaging data is reconstructed and stored in image memory 72, 88, 106 from where it is retrieved by a calibration processor 164. The calibration processor 164 determines a quality assurance (QA) transformation for each scanner 12, 26, 36 based on a difference between an actual coordinate position and an expected coordinate position of the centroid of each fiducial marker 130, 140, or other image structures of the phantom 162.

In an embodiment shown in FIG. 6, the calibration phantom 162 includes a structure which moves the markers 130, 140 relative to each other in a manner that simulates physiological motion. For example, the frame 163 has controlled flexibility or elasticity. A bladder 182 is mounted in the frame. An inflation/deflation device 184 under control of a physiological motion simulation controller 186 cyclically inflates and deflates the bladder to simulate physiologic motion, such as respiratory motion. Other physiological motion simulating structures, such as mechanical mechanisms, a plurality of electro-mechanical actuators, a plurality of pneumatic-mechanical actuators, and the like, are also contemplated.

In another embodiment, the diagnostic imaging system 10 is used for therapy planning procedures, such as radiation therapy planning, ablation therapy planning, interventional procedure planning, or the like. For example, in radiation therapy planning the target region, e.g. a tumor, lesion, or the like, is periodically monitored using one or more of the scanners 12, 26, 36 for changes in shape, size, position, function, etc. These monitored changes can be used by a radiation therapy delivery system to ensure the subject receives a sufficient radiation dose to eradicate the target region without damaging healthy surrounding tissue. The fusion of CT and MR image data acquired in one scanning session with a common patient support, to improve registration, is beneficial for radiation treatment planning or treatment monitoring follow up purposes.

In another embodiment, the entire multiple modality imaging system 10 as illustrated in FIG. 1 is disposed within or mounted on a mobile vehicle for transportation within a medical facility, between medical facilities, an off-site facility, or the like. For example, the system 10 can be stored in and transported by a large truck trailer which can be moved from one location to another to serve as a full-service medical imaging facility.

A method of making a multiple modality marker 130, 140, 150 includes providing a first portion 132 comprising of a radioisotope which is imageable by the nuclear imaging scanner 26 and the CT scanner 36. The first portion 132 is surrounded with a second portion 134 comprising of a flexible material which is imageable by a MR scanner 12 and the CT scanner 36. The first and second portions 132, 134 are surrounded by a housing 136, particularly acrylic, which provides support.

With reference with FIG. 6, a method of using multiple modality imaging system 10 is presented. The scanner comprises an MR scanner 12 which defines an MR imaging region 18, a nuclear imaging scanner 26 which defines a nuclear imaging region 34, and an CT 36 scanner which defines an CT imaging region. The method includes fixating the calibration phantom 162, which comprises a plurality of markers 130, 140, 150 that are supported by the common frame 163, to the common patient support 46 (S100). The phantom 162 is moved into in each of the MR, nuclear, and CT imaging regions 18, 34, 42 and image data is acquired therefrom (S102). At least one QA transformation is determined (S104) based on a coordinate position of a centroid of each of the plurality of markers 130, 140, 150 for each scanner 12, 26, 36. The subject is positioned on the common patient support (S106) which moves linearly through the MR, nuclear, and CT imaging regions 18, 34, 42. The subject or an accessory 200 attached to the subject is fitted with at least one marker 130, 140, 150 (S108) which is imageable by each of the MR, nuclear, and CT scanners 12, 26, 36. The subject is moved linearly into the MR imaging region 18 and MR image data is acquired (S110) therefrom. The subject is moved linearly into the nuclear imaging region 34 and nuclear image data is acquired (S110) therefrom. The subject is moved linearly into the CT imaging region 42 and CT image data is acquired (S110) therefrom. The order of which the image data is acquired is arbitrary. However, workflow can be taken into consideration when determining the order. The acquired image data of the subject is reconstructed into an MR, nuclear, and CT image representation according to the at least one QA transformation (S112). The reconstructed image representations are aligned or registered to one another according to the at least one marker 130, 140, 150 fitted to the subject, the patient support 46, and an accessory attached to subject (S114).

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A multiple modality imaging system, comprising:
a magnetic resonance (MR) scanner which defines an MR imaging region which receives a subject along an MR longitudinal axis;
a nuclear imaging scanner which defines a nuclear imaging region which receives the subject along a nuclear longitudinal axis, the nuclear longitudinal axis being aligned with the MR longitudinal axis;
an computed tomography (CT) scanner which defines an CT imaging region which receives the subject along an CT longitudinal axis, the CT longitudinal axis being aligned with the MR and nuclear longitudinal axes; and
a common patient support which moves linearly through the MR, nuclear, and CT imaging regions.

2. The multiple modality imaging system according to claim 1, further including at least one marker including:
a radio-isotope marker which is imageable by the nuclear imaging scanner and the computed tomography (CT) scanner;
a magnetic resonance (MR) marker which is imageable by the MR scanner and the CT scanner, the MR marker being composed of a flexible material which surrounds the radio-isotope marker; and
a housing which supports the MR and radio-isotope markers.

3. A marker useable with the multiple modality imaging system of claim 1, the marker comprising:
a radio-isotope marker which is imageable by a nuclear imaging system and a computed tomography (CT) scanner;
a magnetic resonance (MR) marker which is imageable by a magnetic resonance scanner and the CT scanner, the MR marker being composed of a flexible material which surrounds the radio-isotope marker;
a rigid housing which supports and surrounds the MR marker.

4. The marker of claim 2,
wherein a centroid of the radio-isotope marker a centroid of the MR marker have a fixed geometric relationship therebetween.

5. The marker according to Claim 2, wherein the MR marker is a silicone rubber and the radio-isotope marker which is at least one of a solid radioisotope and a liquid encapsulated radio-isotope.

6. The marker according to claim 2, wherein the MR marker is a silicone rubber and the radio-isotope marker is at least one of a solid powder or liquid which is a substantially uniformly dispersed throughout the silicone rubber.

7. A calibration phantom or use with a multiple modality diagnostic image scanner, comprising:
a plurality of markers according to claim 2 supported by a common frame having a known and predictable geometry.

8. The calibration phantom according to claim 7, wherein the markers are arranged in at least one pattern of lines with varying widths, spacings, and orientations.

9. The calibration phantom according to claim 7, further including:
a structure which causes the markers to move relative to each other in a manner that simulates cyclic physiological motion.

10. The multiple modality imaging system according to claim 7, wherein the calibration phantom fixated to the patient support to be moved into and imaged in each of the MR, nuclear, and CT imaging regions; and further including:
a calibration processor which determines at least one quality assurance transformation based on an a coordinate position of a centroid of each of the plurality of markers for each scanner.

11. The multiple modality imaging system according to claim 2, further including:
a fusion processor which combines reconstructed a three-dimensional (3D) image representation of a subject from each of the MR, nuclear, and CT scanners into a composite image representation based on a coordinate position of a centroid of the at least one fiducial marker.

12. The multiple modality imaging system according To claim 2, further including:
at least one accessory attached to the patient which includes a plurality of markers.

13. The multiple modality imaging system according To claim 2, further including:
a gantry track along which the nuclear image scanner and the CT scanner linearly translate to form a closed arrangement between the MR scanner, nuclear scanner, and CT scanner to reduce transit time and distance of the common patient support between the MR, nuclear, and CT imaging regions.

14. The multiple modality imaging system according To claim 2, wherein the CT scanner is a flat panel CT scanner which shares a common gantry with the nuclear image scanner to reduce a footprint of the system.

15. The multiple modality imaging system according to claim 2, wherein the multiple modality imaging system is disposed on a mobile platform which can be transported from one location to another.

16. A method of using multiple modality imaging system comprising an MR scanner which defines an MR imaging region, a nuclear imaging scanner which defines a nuclear imaging region, and an computed tomography (CT) scanner which defines an CT imaging region, the method comprising:
positioning a subject on a common patient support which moves linearly through the MR, nuclear, and CT imaging regions;
moving the subject linearly into the MR imaging region and acquiring MR image data;
moving the subject linearly into the nuclear imaging region and acquiring nuclear image data; and
moving the subject linearly into the CT imaging region and acquiring CT image data.

17. The method according to claim 16, further including:
prior to acquiring image data, fitting the subject with at least one marker useable with each of the MR, nuclear, and CT scanners comprising of a radio-isotope marker which is imageable by a nuclear imaging system and a computed tomography (CT) scanner surrounded by a flexible MR marker which is imageable by a magnetic resonance scanner and the CT scanner;
after acquiring image data, reconstructing the image data into an MR image representation, a nuclear image representation, and an CT image representation respectively; and
aligning the MR, nuclear, and CT image representations according to the fitted at least one marker.

18. The method according to claim 16, further including:
prior to positioning the patient, fixating a calibration phantom comprising a plurality of markers supported by a common frame having a known and predictable geometry to the common patient support;
moving into and acquiring image data of the calibration phantom in each of the MR, nuclear, and CT imaging regions;
determining at least one quality assurance transformation based on a coordinate position of a centroid of each of the plurality of markers for each scanner; and
reconstructing image data acquired from each of the MR, nuclear, and CT scanners according to the at least one quality assurance transformation.

19. An imaging system, comprising:
a magnetic resonance (MR) scanner which defines an MR imaging region;
a nuclear imaging scanner which defines a nuclear imaging region which shares a common longitudinal axis with the MR imaging region;
a flat panel computed tomography (CT) scanner which defines an CT imaging region which shares the common longitudinal axis with the MR imaging region and the CT imaging region;
a common patient support which moves linearly through the MR, nuclear, and CT imaging regions; and
a gantry track along which the nuclear image scanner and the CT scanner linearly translate to form a closed arrangement between the MR scanner, nuclear scanner, and CT scanner to reduce transit time and distance of the common patient support between the MR, nuclear, and CT imaging regions.

20. The imaging system according to claim 19, wherein the nuclear imaging scanner and the flat panel CT scanner share a common gantry to reduce a footprint of the imaging system.
